(19) United States
(12) Patent Application Publication
(10) Pub. No.: US 2021/0346516 A1
(43) Pub. Date: Nov. 11, 2021

(54) NANOHYDROXYAPATITE NANOCARRIER FOR GENETIC CARGO

(71) Applicant: South Dakota Board of Regents, Pierre, SD (US)

(72) Inventors: Bernard W.M. WONE, Vermillion, SD (US); Grigoriy SEREDA, Vermillion, SD (US)

(73) Assignee: South Dakota Board of Regents, Pierre, SD (US)

(21) Appl. No.: 17/316,045

(22) Filed: May 10, 2021

Related U.S. Application Data
(60) Provisional application No. 63/022,164, filed on May 8, 2020.

(51) Int. Cl. A61K 47/69; A61K 47/38; A61K 47/54; C12N 15/82; C12N 15/87; A61K 31/713

(52) U.S. Cl. CPC A61K 47/6929; A61K 47/38; A61K 47/542; A61K 47/6923; C12N 15/87; A61K 31/713; C12N 15/8206

(57) ABSTRACT

The disclosure is directed to nanoparticles, compositions comprising nanoparticles, and methods of using the nanoparticles and compositions to introduce genetic material into a host cell, and in particular, a plant cell nucleus, to cause transformation of the host cell through the expression of genes on the introduced genetic material.

### RELATED APPLICATIONS

This application claims priority under 35 U.S.C. § 119(e) to U.S. Provisional Patent Application No. 63/022,164, filed May 8, 2020, which is incorporated herein by reference.

### BACKGROUND OF THE INVENTION

Plant genetic engineering will be crucial for deciphering the genetic of basis of complex traits, furthering advances in crop genomics, and enabling plant-based production of recombinant pharmaceutical proteins. Development of a biocompatible transformation system for passive delivery of genetic cargo into a variety of plant species will not only aid in directly testing and deciphering gene function in a variety of plant species but will also facilitate enhanced crop productivity and allow scale-up of the production of recombinant proteins via genome manipulation. Increasing crop productivity and environmental stress resistance will be essential to meet the growing food and energy demands of a burgeoning human population under increasingly stressful environmental conditions. However, plant genetic engineering depends upon the delivery of genes across the plant cell wall using established methods of genetic transformation that have many disadvantages.

Transformation represents a bottleneck for genetic manipulation of plants because of the plant cell wall. Established plant genetic transformation methods rely heavily on Agrobacterium to mediate transformation, even though this approach is species limited. In addition, DNA from Agrobacterium remains in the plant host cells, randomly integrates DNA sequences into the plant genome, often resulting in variable transgene expression or insertional mutagenesis. Although genome editing is more precise and provides novel opportunities to directly determine gene function or manipulate the genome, it still relies on genetic transformation methods that are also fraught with limitations. Specifically, introducing the CRISPR/Cas9 toolbox into plant cells still depends on Agrobacterium-mediated transformation to integrate gene editing components into the plant genome and can still result in undesirable off-target effects and insertional mutations in the genome. Similarly, virus component-mediated transformation of DNA plasmids into plants is species-limited, integrates viral DNA into the host genome, and limits genetic cargo size. Furthermore, this conventional method is subjected to regulatory oversight because of the pathogenic origins of these vectors. Although biolistic DNA plasmid delivery is an efficient method of genetic transformation that does not require Agrobacterium or vectors derived from bacteria or viruses, it can result in high frequencies of random plasmid or chromosomal DNA fragment insertion as well as physical injury to the plant.

More recently, the use of engineered nanomaterials as gene nanocarriers has shown great promise because nanoparticles can passively enter plant tissues including stems, leaves, roots, and seeds. Because of this passive ability to traverse the cell wall and plasma membrane, nanoparticle-mediated delivery of genetic cargo shows great promise for advancing plant genetic engineering.6 In addition, nanoparticles have high DNA binding ability, high transformation efficiency, and without genome integration. Although single-wall carbon nanotubes could be used for such an approach, their major disadvantage for gene introduction is that carbon-based or other highly persistent nanomaterials do not readily break down in organisms or in the environment and can disrupt cellular functions. Specifically, carbon nanomaterials including nanotubes, fullerenes, and graphenes are extremely stable and difficult to degrade and thus can be harmful or persist in plant and animal cells or in the environment. Most importantly, using highly persistent carbon-based nanomaterials to carry DNA plasmids or gene-editing components increases the risk of unwanted and unintended horizontal transfer of heterologous genes to other organisms.

The present invention addresses these needs by using advanced optimizable, biodegradable, and biocompatible nanohydroxyapatite nanoparticles that function as a nucleic acid delivery vehicle into plants in order to, for example, assay the genomic basis of complex traits such as abiotic stress tolerance and to modify the genomes of crop plants to improve stress tolerance.

### SUMMARY OF THE INVENTION

The development of an ideal nanocarrier that is both readily biodegradable and biocompatible has been long awaited in the field of plant transformation. Although biodegradable calcium-based mineral nanoparticles such as calcium phosphate (CaP) have been successfully tested in plants, further explorations of the use of CaP nanoparticles for plant transformation have not yet been further published to our knowledge. The CaP nanoparticles describe a general type of particle with various Ca/P ratios and acidities. The use of nanoHAs (nHAs) as novel gene carriers has so far been demonstrated in animal cells, bacterial cells, and yeast cells, but their effectiveness for gene delivery in plants has yet to be shown. More recently, biocompatible nHAs have been successfully used in biomedical systems to deliver diverse molecular cargo and in agricultural systems as synthetic fertilizer. Most importantly, synthetic nHAs have many advantages over single-wall carbon nanotubes as gene carriers for plants because they are noncytotoxic and nonecotoxic, readily broken down and used by plants as nutrients and can be harmlessly assimilated by either plant or animal cells or the environment. Therefore, nHAs can be considered as nano-biomimetic gene carriers.

The disclosure reports the use of biocompatible and optimizable nHA-based DNA carriers to passively (or with vacuum assistance) deliver DNA plasmids for genetic transformation in planta to further advance the nanoparticle-mediated transformation approach. We have synthesized and validated a system for highly efficient, passive, and harmless genetic transformation of both model and crop plants. We have used nHAs functionalized with arginine for transient in vivo infiltration of plasmid-nHA conjugates into leaves of tobacco (Nicotiana benthamiana), Arabidopsis thaliana, and common ice plant (Mesembryanthemum crystallinum) and have observed protein expression from introduced transgenes. We have also shown strong protein expression in germinating seeds of field mustard (Brassica rapa), wheat (Triticum aestivum), and barley Hordeum vulgare L.) after incubation in a solution of plasmid-nHA conjugates. The present study demonstrates highly efficient, passive, and harmless delivery of genetic cargo with less potential for horizontal transfer of recombinant genes by the nanocarrier. This combination of highly desirable features is not achievable with either current nanoparticle-mediated or established transformation approaches. Altogether, the nano-biomimetic transformation system will revolutionize plant genetic engineering, the ability to investigate the genomic basis of traits, and the ability to manipulate the genomes of a wide variety of plants for many possible biotechnology applications.

Accordingly, the disclosure is directed to nanoparticles, compositions comprising nanoparticles, and methods of use to introduce genetic material into a plant nucleus and cause transformation of the plant cell through the expression of genes on the introduced genetic material.

Certain embodiments of the invention provide a hydroxyapatite nanoparticle and a polynucleotide attached to a surface of the hydroxyapatite nanoparticle.

Preferably, the hydroxyapatite nanoparticle is about wherein the nanoparticle is rod-shaped, having an average diameter of about 2 nm to about 20 nm and an average length of about 20 nm to about 70 nm.

In some embodiments, the rod-shaped nanoparticle consisting of hydroxyapatite; a positively charged amino acid disposed on a surface of the hydroxyapatite; and a polynucleotide ionically conjugated to the positively charged amino acid, wherein the nanoparticle is about 8.5 nm to about 15 nm in diameter and about 42 nm to about 55 nm in length.

Other embodiments include a composition comprising: a rod-shaped hydroxyapatite nanoparticle and a polynucleotide attached to a surface of the hydroxyapatite nanoparticle, and a pharmaceutically acceptable carrier; wherein the hydroxyapatite nanoparticle is about 8.5 nm to about 15 nm in diameter and about 42 nm to about 55 nm in length.

Embodiments of the invention include one or polynucleotides attached to the surface of the hydroxyapatite nanoparticle through ionic interaction. The polynucleotide may be, for example, a circular piece of nucleic acid (e.g., a plasmid) or a linear price of nucleic acid. Preferably, the polynucleotide comprises one or more genes including control sequences that may allow the gene to be expressed in a cell, and in particular, a plant cell. The polynucleotide may be about 0.1 kb to about 15 kb in length, about 1 kb to about 15 kb in length, about 1 kb to about 14 kb in length, about 5 kb to about 14 kb in length, and preferably, about 14 kb in length.

In some embodiments, the surface of the hydroxyapatite nanoparticle is functionalized with a linker, such as a positively charged amino acid, wherein the polynucleotide is ironically attached to the linker. In some embodiments, the linker is an arginine amino acid or derivative thereof, and the polynucleotide is ionically bonded to the arginine amino acid.

The disclosure also provides for methods of genetically transforming a cell comprising contacting the cell with a nanoparticle of as disclosed herein, such that the nanoparticle passes through a cell membrane of the cell and is transported to a nucleus, and a heterologous gene of the polynucleotide is expressed to transform the cell.

Other embodiments include a method for producing a genetically modified plant comprising contacting a plant, a plant cell, a plant seed, or plant tissue with a nanoparticle functionalized with a polynucleotide, the nanoparticle comprising a rod-shaped hydroxyapatite core particle and the polynucleotide is ionically bonded to a surface of the nanoparticle, and the nanoparticle passes through a cell wall of the plant cell and is transported to a plant nucleus such that a heterologous gene of the polynucleotide is expressed.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Morphologies of synthesized nanohydroxyapatites (nHAs). (a) Scanning Electron Microscope (SEM) image of nHAs. (b) Transmission Electron Microscope (TEM) image of nHAs. (c) TEM image of nHAs functionalized with arginine. Scale bars: 100 nm (a); and 20 nm (b-c). (d) Particle diameter (nm) distribution of nHAs and R-nHAs. (e) Particle length (nm) distribution of nHAs (red colored columns) and RnHAs.

FIG. 2. Physicochemical characteristics of nanohydroxyapatites (nHAs). Powdered x-ray diffraction (PXRD) pattern of nHAs and R-nHAs (functionalized with arginine).

FIG. 3. Conjugation of DNA plasmid (pDNA) with nanohydroxyapatites (nHAs). (a) Conjugation efficiency assay of pDNA and nHAs by measuring the optical density of the supernatant (i.e., unbound pDNA in an aqueous solution of water) at 260 nm indicates the 1:200 mass ratio conjugates have the highest conjugating efficiencies. Error bars are standard error of the mean (n=3). (b) Agarose gel electrophoretogram of pDNA-R-nHA conjugates confirms the 1:200 mass ratio conjugates is the most efficient ratio as indicated by the bright intensity of the band in lane 6. Lane 1—1:10 mass ratio. Lane 2—1:30 mass ratio. Lane 3—1:50 mass ratio. Lane 4—1:70 mass ratio. Lane 5—1:100 mass ratio. Lane 6—1:200 mass ratio. Lane 7—pDNA. Faint bands indicate relaxed and supercoiled (bright bands) conformations of pDNA or pDNA-nHA conjugates. (c) DNase assay of conjugates suggests nHAs might reduce or inhibit endonuclease activity, especially relaxed forms of the plasmid. Lane 1—pDNA-R-nHA conjugates with DNase treatment. Lane 2—pDNA-R-nHA conjugates without DNase treatment. Lane 3—pDNA-nHA conjugates with DNase treatment. Lane 4—pDNA-nHA conjugates without DNase treatment. Lane 5—pDNA with DNase treatment. Lane 6—pDNA without DNase treatment. Faint bands indicate relaxed and supercoiled (bright bands) conformations of pDNA or pDNA-nHA conjugates.

FIG. 4. Plasmid DNA (pDNA) delivery into mature leaves with nanohydroxyapatites (nHAs) and subsequent GUS expression. (a) Model of uptake and translocation of DNA-nHA conjugates and subsequent expression of introduced gene(s) in a plant cell. Dotted-line arrows represent trafficking of complexes, whereas solid-line arrows represent expression of introduced gene(s).DNA-nHA—plasmids or PCR amplicons conjugated to nHA. ML—middle lamella. PCW primary cell wall. SCW—secondary cell wall. PM—plasma membrane. nDNA—nuclear DNA. mRNA—messenger RNA. GUS—stained GUS protein product. GFP—GFP product. (b-h). Transient GUS expression in leaves of mature tobacco (Nicotiana benthamiana) and Arabidopsis thaliana after in vivo infiltration of pDNA-R-nHA conjugates of healthy plants using a syringe, and Arabidopsis, and ice plant (Mesembryanthemum crystallinum) after vacuum infiltration of pDNA-R-nHA conjugates in detached leaves from healthy plants: (b) Control (untreated)—tobacco; (c) Agrobacterium-mediated expression of GUS in tobacco; (d) pDNA-R-nHA-mediated expression of GUS in tobacco; (e) Control (untreated—infiltrated with water)—Arabidopsis; (f) pDNA-R-nHAmediated expression of GUS in Arabidopsis; (g) Control (untreated—infiltrated with water)—ice plant; and (h) pDNA-R-nHA-mediated expression of GUS in ice plant. Blue spots or patches (arrows) represent histochemical staining of GUS.

FIG. 5. Plasmid DNA (p(DNA) delivery into seeds with arginine-functionalized nanohydroxyapatites (R-nHAs) via incubation in a solution of pDNA-R-nHA conjugates and subsequent GUS expression. (a) Control (untreated—incubated in water)—barley (Hordeum vulgare) seed. (b) Transient GUS expression in imbibed barley seed after incubation in a solution of pDNAR—nHA conjugates. (c) Magnification at 25× of the barley seedling. (d) Control (untreated—incubated in water)—wheat (Triticum aestivum) seed. (e) Transient GUS expression in imbibed wheat seed after incubation in a solution of pDNA-R-nHA conjugates. Arrow indicates embryonic tissue expressing GUS. (f) Control (untreated—incubated in water)—Brassica rapa seed at 8× magnification. (g) Transient GUS expression in imbibed Brassica rapa seed after incubation in a solution of pDNA-R-nHA conjugates. Magnification at 8× of developing seedling. h. Magnification at 15× of the seedling radical. Blue spots or patches (arrows) represent histochemical staining of GUS in all the panels.

FIG. 6. Delivery of plasmid DNA (pDNA) with green fluorescent protein gene into root cells of seedlings with arginine-functionalized nano-hydroxyapatites (R-nHAs) via incubation in a solution of pDNA-R-nHA conjugates and subsequent GFP expression. (a) Root cells of 4 d old Arabidopsis seedling after incubation in water (i.e., control). (b) Transient GFP expression in imbibed root cells of 4 d Arabidopsis seedling after incubation in a solution of pDNA-R-nHA conjugates. (c) Root cells of 4 d field mustard (Brassica rapa) seedling after incubation in water (i.e., control). (d) Transient GFP expression in imbibed root cells of 4 d field mustard seedling after incubation in a solution of pDNA-R-nHA conjugates. Green patches (arrows) indicate GFP protein expression in cytoplasm of root cells in panels (b) and (d), whereas the green outline of the cell is due to autofluorescence of lignin in the cell wall in all panels. All images are at 100 magnification. Scale bars: 20 mm.

### DETAILED DESCRIPTION

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The term “about” can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, composition, or embodiment. The term about can also modify the end-points of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect. An appropriate “effective” amount in any individual case may be determined using techniques, such as a dose escalation study.

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

Nucleic acids or polynucleotides refer to RNA or DNA that is linear or branched, single or double stranded, or a hybrid thereof. The term also encompasses RNA/DNA hybrids. These terms also encompass untranslated sequence located at both the 3′ and 5′ ends of the coding region of the gene: at least approximately 1000 nucleotides of sequence upstream from the 5′ end of the coding region and at least approximately 200 nucleotides of sequence downstream from the 3′ end of the coding region of the gene. Less common bases, such as inosine, 5-methylcytosine, 6-methyladenine, hypoxanthine and others can also be used for antisense, dsRNA and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. The antisense polynucleotides and ribozymes can consist entirely of ribonucleotides or can contain mixed ribonucleotides and deoxyribonucleotides. The polynucleotides of the invention may be produced by any means, including genomic preparations, cDNA preparations, in vitro synthesis, RT-PCR, and in vitro or in vivo transcription.

As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” in the context of a peptide indicates that a peptide comprises a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even 95%, 96%, 97%, 98% or 99%, sequence identity to the reference sequence over a specified comparison window. In certain embodiments, optimal alignment is conducted using the homology alignment algorithm of Needleman and Wunsch (Needleman and Wunsch, JMB, 48, 443 (1970)). An indication that two peptide sequences are substantially identical is that one peptide is immunologically reactive with antibodies raised against the second peptide. Thus, a peptide is substantially identical to a second peptide, for example, where the two peptides differ only by a conservative substitution. Thus, the invention also provides nucleic acid molecules and peptides that are substantially identical to the nucleic acid molecules and peptides presented herein.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As used herein, the term “transgene” refers to a DNA molecule artificially incorporated into an organism's genome as a result of human intervention, such as a plant transformation method. As used herein, the term “transgenic” means comprising a transgene, for example a “transgenic plant” refers to a plant comprising a transgene in its genome and a “transgenic trait” refers to a characteristic or phenotype conveyed or conferred by the presence of a transgene incorporated into the plant genome. As a result of such genomic alteration, the transgenic plant is something distinctly different from the related wild-type plant and the transgenic trait is a trait not naturally found in the wild-type plant. Transgenic plants of the invention comprise the recombinant DNA molecules and engineered proteins provided by the invention.

As used herein, the term “heterologous” refers to the relationship between two or more things derived from different sources and thus not normally associated in nature. For example, a protein-coding recombinant DNA molecule is heterologous with respect to an operably linked promoter if such a combination is not normally found in nature. In addition, a particular recombinant DNA molecule may be heterologous with respect to a cell, seed, or organism into which it is inserted when it would not naturally occur in that particular cell, seed, or organism.

As used herein, a “DNA construct” is a recombinant DNA molecule comprising two or more heterologous DNA sequences. DNA constructs are useful for transgene expression and may be comprised in vectors and plasmids. DNA constructs may be used in vectors for transformation, that is the introduction of heterologous DNA into a host cell, to produce transgenic plants and cells, and as such may also be contained in the plastid DNA or genomic DNA of a transgenic plant, seed, cell, or plant part. As used herein, a “vector” means any recombinant DNA molecule that may be used for plant transformation. DNA molecules as set forth in the sequence listing, can, for example, be inserted into a vector as part of a construct having the DNA molecule operably linked to a gene expression element that functions in a plant to affect expression of the protein encoded by the DNA molecule. Methods for constructing DNA constructs and vectors are well known in the art. The components for a DNA construct, or a vector comprising a DNA construct, generally include one or more gene expression elements operably linked to a transcribable DNA sequence, such as the following: a promoter for the expression of an operably linked DNA, an operably linked protein-coding DNA molecule, and a 3′ untranslated region. Gene expression elements useful in practicing the present invention include, but are not limited to, one or more of the following type of elements: promoter, 5′ untranslated region, enhancer, leader, cis-acting element, intron, 3′ untranslated region, and one or more selectable marker transgenes.

The DNA constructs of the invention may include a promoter operably linked to a protein-coding DNA molecule provided by the invention, whereby the promoter drives expression of the heterologous protein molecule. Promoters useful in practicing the present invention include those that function in a cell for expression of an operably linked polynucleotide, such as a bacterial or plant promoter. Plant promoters are varied and well known in the art and include those that are inducible, viral, synthetic, constitutive, temporally regulated, spatially regulated, and/or spatio-temporally regulated.

### Embodiments of the Invention

Certain embodiments of the invention include a nanoparticle comprising hydroxyapatite and a polynucleotide attached to the surface of the hydroxyapatite nanoparticle. Hydroxyapatite (HA) is a form of calcium phosphate with a specific Ca to P ratio, presence of protons or hydroxyls, and a crystalline form or lack of the crystalline form (an amorphous state). Specifically, hydroxyapatite is Ca₁₀(PO₄)₆(OH)₂, which can be presented as a compound of 3Ca₃(PO₄)₂ and Ca(OH)₂ and includes a molar ratio of Ca to P of 1.7. While CaP nanoparticles are a more general type of particle with various Ca/P ratios and acidities, HA is the most common form of CaP present in mammalian bones and teeth. Typically, the nanoparticles of the present disclosure are much smaller in diameter than the CaP nanoparticles and, unexpectedly, have high efficacy in transforming explants.

The hydroxyapatite nanoparticle includes a size and shape configured to pass through the cell wall of a plant as well as through the plant nuclear pore. Thus, the nanoparticle may be of any size and shape to facilitate such action. Preferably, the nanoparticle is rod-shaped. Preferably, the rod-shaped hydroxyapatite nanoparticle has an average aspect ratio greater than 1, and more preferably, the average aspect ratio is 5 or greater, and more preferably 10 or greater. The aspect ratio (L/D) of an object is defined as the ratio of its longest dimension to shortest dimension. For rod-shaped particles, the aspect ratio is simply the ratio of the length to the diameter (L/D).

Alternatively, hydroxyapatite nanoparticle is needle shaped, spherical shaped, or a combination thereof. In other embodiments of the invention, the nanoparticle may be pocket-shaped, having a concave side that could protect plasmids from degradation.

As noted, the hydroxyapatite nanoparticle is sized and shaped to passively pass through a plant cell wall and nuclear pore. Preferably, the hydroxyapatite nanoparticle is a rod-shaped particle of about 5 nm to about 100 nm in diameter, 5 nm to about 90 nm in diameter, about 5 nm to about 80 nm in diameter, about 5 nm to about 60 nm in diameter, about 5 nm to about 50 nm in diameter, about 5 nm to about 40 nm in diameter, about 5 nm to about 30 nm in diameter, about 5 nm to about 20 nm in diameter, or about 5 nm to about 10 nm in diameter.

In some embodiments, the average length of a rod-shaped hydroxyapatite nanoparticle is about 5 nm to about 150 nm, about 5 nm to about 140 nm, about 5 nm to about 130 nm, about 5 nm to about 120 nm, about 5 nm to about 110 nm, about 5 nm to about 100 nm, about 5 nm to about 90 nm, about 5 nm to about 80 nm, about 5 nm to about 70 nm, about 5 nm to about 60 nm, about 5 nm to about 50 nm, about 5 nm to about 45 nm, about 5 nm to about 40 nm, about 5 nm to about 35 nm, about 5 nm to about 30 nm, about 5 nm to about 25 nm, or about 5 nm to about 20 nm.

In some embodiments, the rod-shaped hydroxyapatite nanoparticle has an average diameter of about 2 nm to about 20 nm and an average length of about 20 nm to about 70 nm. In some embodiments, the rod-shaped hydroxyapatite nanoparticle has an average diameter about 5 nm to about 15 nm and the average length is about 35 nm to about 55 nm. In another embodiment, the rod-shaped hydroxyapatite nanoparticle has an average diameter of about 7 nm to 12 nm and the average length is about 40 nm to about 50 nm. In another embodiment, the rod-shaped hydroxyapatite nanoparticle has the average diameter is about 8.5 nm and the average length is about 42 nm. In other embodiments, the average diameter of a rod-shaped hydroxyapatite nanoparticle is about 2 nm to about 5 nm.

In some embodiments, the surface of the hydroxyapatite nanoparticle is functionalized to include a positively charged linker. In some embodiments, the positively charged linker comprises one or more amino acids, preferably one or more arginine amino acids or one or more lysine amino acids, and more preferably, one or more arginine amino acids. In some embodiments, the linker comprises or consists of arginine and/or lysine amino acids. In some embodiments, the linker consists of one or more arginine amino acids. In some embodiments, the polynucleotide is ionically bonded to the linker (e.g., an arginine linker). Other suitable linkers include those having a carboxyl or phosphate functional group that can bind to the surface of the hydroxyapatite nanoparticle and a positively charged function group (e.g., amino or guanidinium group) to bind to the polynucleotide.

In some embodiments, functionalizing the surface of the hydroxyapatite may increase the diameter and length of the rod-shaped nanoparticles. Accordingly, in some embodiments, a functionalized hydroxyapatite nanoparticle may have an average diameter of about 2 nm to about 20 nm and an average length of about 20 nm to about 70 nm, an average diameter of about 10 nm to about 18 nm and an average length of about 45 nm to about 60 nm, an average diameter of about 12 nm to about 17 nm and an average length of about 50 nm to about 57 nm, or an average diameter of about 15 nm and an average length of about 55 nm.

Embodiments of the invention comprise a polynucleotide bonded to the hydroxyapatite surface. In some embodiments, the polynucleotide is attached to the surface of the hydroxyapatite through ionic interaction, and more preferably, the polynucleotide is bonded to a linker on the surface of the hydroxyapatite where the linker is directly attached to the surface of the hydroxyapatite.

Preferably, the polynucleotide that is attached to the surface of the hydroxyapatite is about 0.1 kb to about 25 kb in length, about 0.1 kb to about 20 kb in length, about 0.1 kb to about 15 kb in length, about 1 kb to about 15 kb in length, about 5 kb to about 15 kb in length, about 10 kb to about 15 kb in length, or about 12 kb to about 14 kb in length. Preferably, the polynucleotide is a circular piece of nucleic acid such as a plasmid. Alternatively, the polynucleotide may be linear.

In some embodiments, the polynucleotide is attached to the surface of the nanohydroxyapatite nanoparticle in various mass ratios (w:w) ranging from about 1:5 to about 1:300 polynucleotide:nanoparticle. In other embodiments, the mass ratio of polynucleotide to nanoparticle is about 1:10 to about 1:250, or about 1:20 to about 1:200, or about 1:30 to about 1:200, or about 1:40 to about 1:200, or about 1:50 to about 1:200, or about 1:60 to about 1:200, or about 1:70 to about 1:200, or about 1:80 to about 1:200, or about 1:90 to about 1:200, or about 1:100 to about 1:200, or about 1:150 to about 1:200. In some embodiments, Polynucleotides are only on the surface of the nanohydroxyapatite (i.e., excluded from the interior of the nanoparticle).

Polynucleotides for use with the hydroxyapatite nanoparticle may include, for example, expression vectors. Expression vectors include at least one genetic marker operably linked to a regulatory element (for example, a promoter) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are well known in the transformation arts, and include, for example, genes that code for enzymes that metabolically detoxify a selective chemical agent which may be an antibiotic or an herbicide, or genes that encode an altered target which is insensitive to the inhibitor. A few positive selection methods are also known in the art.

One commonly used selectable marker gene for plant transformation is the neomycin phosphotransferase II (nptII) gene which, when under the control of plant regulatory signals, confers resistance to kanamycin. (Fraley et al., Proc. Natl. Acad. Sci. USA, 80:4803 (1983)). Another commonly used selectable marker gene is the hygromycin phosphotransferase gene which confers resistance to the antibiotic hygromycin. (Vanden Elzen et al., Plant Mol. Biol., 5:299 (1985)).

Additional selectable marker genes of bacterial origin that confer resistance to antibiotics include gentamycin acetyl transferase, streptomycin phosphotransferase and aminoglycoside-3′-adenyl transferase, the bleomycin resistance determinant (Hayford et al., Plant Physiol., 86:1216 (1988); Jones et al., Mol. Gen. Genet., 210:86 (1987); Svab et al., Plant Mol. Biol., 14:197 (1990); Hille et al., Plant Mol. Biol., 7:171 (1986)). Other selectable marker genes confer resistance to herbicides such as glyphosate, glufosinate, or bromoxynil (Comai et al., Nature, 317:741-744 (1985); Gordon-Kamm et al., Plant Cell, 2:603-618 (1990); Stalke et al., Science, 242:419-423 (1988)).

Selectable marker genes for plant transformation not of bacterial origin include, for example, mouse dihydrofolate reductase, plant 5-enolpyruvylshikimate-3-phosphate synthase, and plant acetolactate synthase (Eichholtz et al., Somatic Cell Mol. Genet., 13:67 (1987); Shah et al., Science, 233:478 (1986); Charest et al., Plant Cell Rep., 8:643 (1990)).

Another class of marker genes for plant transformation requires screening of presumptively transformed plant cells, rather than direct genetic selection of transformed cells, for resistance to a toxic substance such as an antibiotic. These genes are particularly useful to quantify or visualize the spatial pattern of expression of a gene in specific tissues and are frequently referred to as reporter genes because they can be fused to a gene or gene regulatory sequence for the investigation of gene expression. Commonly used genes for screening presumptively transformed cells include β-glucuronidase (GUS), β-galactosidase, luciferase, and chloramphenicol acetyltransferase (Jefferson, R. A., Plant Mol. Biol. Rep., 5:387 (1987); Teeri et al., EMBO J., 8:343 (1989); Koncz et al., Proc. Natl. Acad. Sci. USA, 84:131 (1987); DeBlock et al., EMBO J., 3:1681 (1984)).

In vivo methods for visualizing GUS activity that do not require destruction of plant tissue are available (Molecular Probes, Publication 2908, IMAGENE GREEN, pp. 1-4 (1993); Naleway et al., J. Cell Biol., 115:151a (1991)). More recently, a gene encoding Green Fluorescent Protein (GFP) has been utilized as a marker for gene expression in prokaryotic and eukaryotic cells (Chalfie et al., Science, 263:802 (1994)). GFP and mutants of GFP may be used as screenable markers.

In other embodiments, the polynucleotides may comprise the CRISPR-Cas 9/12 system. CRISPR/Cas and similar gene targeting systems are well known in the art with reagents and protocols readily available (Mali et al., (2013), Science, 339(6121), 823-826; Hsu et al., (2014), Cell, 157.6: 1262-1278.). Exemplary genome editing protocols are described in Doudna, and Mali, “CRISPR-Cas: A Laboratory Manual” (2016) (CSHL Press, ISBN: 978-1-621821-30-4) and Ran, F. Ann, et al. Nature Protocols (2013), 8 (11): 2281-2308, and in U.S. Pat. No. 8,697,359 and U.S. Pat. Pub. No. 20180371487. Other exemplary genes of interest include pest resistance, drought resistance, and heat resistance genes as disclosed, for example, in PCT. Pub. No. WO2013169802.

Methods for analyzing a gene integration into the plant genome are known to the person skilled in the art and comprise, but are not limited to polymerase chain reaction (PCR), including inter alia real time quantitative PCR, multiplex PCR, RT-PCR, nested PCR, analytical PCR and the like, microscopy, including bright and dark field microscopy, dispersion staining, phase contrast, fluorescence, confocal, differential interference contrast, deconvolution, electron microscopy, UV microscopy, IR microscopy, scanning probe microscopy, the analysis of plant or plant cell metabolites, RNA analysis, proteome analysis, functional assays for determining a functional integration, e.g. of a marker gene or a transgene of interest, or of a knock-out, Southern-Blot analysis, sequencing, including next generation sequencing, including deep sequencing or multiplex sequencing and the like, and combinations thereof.

One embodiment of the invention includes a nanoparticle comprising a hydroxyapatite core and a polynucleotide attached to a surface of the hydroxyapatite core, wherein the nanoparticle is rod-shaped, having an average diameter of about 2 nm to about 20 nm and an average length of about 20 nm to about 70 nm.

One embodiment of the invention includes a nanoparticle consisting of a hydroxyapatite core and a polynucleotide attached to a surface of the hydroxyapatite core, wherein the nanoparticle is rod-shaped, having an average diameter of about 2 nm to about 20 nm and an average length of about 20 nm to about 70 nm.

In one embodiment, the nanoparticle comprises a hydroxyapatite core, a positively charged amino acid disposed on at least a portion of a surface of the hydroxyapatite core particle, and a polynucleotide ionically conjugated to the positively charged amino acid, wherein the hydroxyapatite core is rod-shaped and is about 8.5 nm to about 15 nm in diameter and about 42 nm to about 55 nm in length.

In one embodiment, the nanoparticle consists of a hydroxyapatite core, a positively charged amino acid disposed on at least a portion of a surface of the hydroxyapatite core particle, and a polynucleotide ionically conjugated to the positively charged amino acid, wherein the hydroxyapatite core is rod-shaped and is about 8.5 nm to about 15 nm in diameter and about 42 nm to about 55 nm in length.

In another embodiment, the nanoparticle consists of a hydroxyapatite core. In another embodiment, the nanoparticle consists of a hydroxyapatite core and a functionalize surface. In other embodiments, the hydroxyapatite nanoparticle may be encapsulated in a lipid shell, such as in a liposome (Karny, et al., Sci Rep 8, 7589 (2018)).

The disclosure also provides for compositions of hydroxyapatite nanoparticles that include one or more pharmaceutically acceptable carrier. The pharmaceutically acceptable carrier may include one or more of water, distilled water, and optionally a thickening agent, one or more buffers, salts or a combination there of.

In some embodiments, a composition that includes nHAs may comprises water and a viscosity thickener such as a cellulose derivative. Exemplary cellulose derivatives include, but are not limited to hydroxyethyl cellulose, methyl cellulose, carboxymethyl cellulose, hydroxypropylmethylcellulose present in a composition (w/v) of about 1%, about 0.9%, about 0.8%, about 0.7%, about 0.6%, about 0.5%, about 0.4%, about 0.3%, about 0.2%, or about 0.1%. In some embodiments, the composition comprises about 0.1% to about 0.8% carboxymethyl cellulose, or about 0.2% to about 0.6% carboxymethyl cellulose, or about 0.5% carboxymethyl cellulose.

Accordingly, one embodiment of the invention is a composition comprising a nanoparticle comprising a hydroxyapatite core and a polynucleotide attached to a surface of the hydroxyapatite core, and a pharmaceutically acceptable carrier, wherein the nanoparticle is rod-shaped, having an average diameter of about 2 nm to about 20 nm and an average length of about 20 nm to about 70 nm.

One embodiment of the invention is a composition comprising a nanoparticle consisting of a hydroxyapatite core and a polynucleotide attached to a surface of the hydroxyapatite core, and a pharmaceutically acceptable carrier, wherein the nanoparticle is rod-shaped, having an average diameter of about 2 nm to about 20 nm and an average length of about 20 nm to about 70 nm.

In one embodiment, a composition comprises a nanoparticle comprising a hydroxyapatite core, a positively charged amino acid disposed on at least a portion of a surface of the hydroxyapatite core particle, a polynucleotide ionically conjugated to the positively charged amino acid, and a pharmaceutically acceptable carrier, wherein the hydroxyapatite core is rod-shaped and is about 8.5 nm to about 15 nm in diameter and about 42 nm to about 55 nm in length.

In one embodiment, the composition comprises a nanoparticle consisting of a hydroxyapatite core, a positively charged amino acid disposed on at least a portion of a surface of the hydroxyapatite core particle, and a polynucleotide ionically conjugated to the positively charged amino acid, wherein the hydroxyapatite core is rod-shaped and is about 8.5 nm to about 15 nm in diameter and about 42 nm to about 55 nm in length.

Certain embodiments of the invention also provide a composition that may be used to introduce genetic material into a host cell such as a mammalian cell, a fungal cell, or a plant cell.

One embodiment provides for a method of introducing a polynucleotide into mammalian cell, a fungal cell, or a plant cell, comprising contacting the cell with any of the hydroxyapatite nanoparticles or compositions disclosed herein such that the nanoparticle passes through the cell membrane of the cell and is transported to the cell nucleus, and a heterologous gene of the polynucleotide is expressed to transform the cell.

In other embodiments, a method for producing a genetically modified plant is disclose comprising contacting a plant, a plant cell, a plant seed, or plant tissue with a nanoparticle functionalized with a polynucleotide, the nanoparticle comprising a rod-shaped hydroxyapatite core particle and the polynucleotide is ionically bonded to a surface of the nanoparticle, and the nanoparticle passes through a cell wall and cell membrane of the plant cell and is transported to a plant nucleus such that a heterologous gene of the polynucleotide is expressed.

In some embodiments, the hydroxyapatite nanoparticles described herein may be used to transform certain plant species traditionally resistant to transformation such as soybean (Glycine max).

In some embodiments, a hydroxyapatite nanoparticle and/or a composition thereof may be vacuum infiltrated into the plant, plant cell, plant seed, or plant tissue as described in (Leuzinger et al., J. Visualized Exp., 2013, 77, 50521). In preferred embodiments of the invention, the nanoparticle or compositions including the nanoparticle may be used to introduce genetic material into a plant, plant cell, plant seed, or other plant issue from a dicotyledonous or a monocotyledonous plant. Exemplary plant, plant cell, seed, or tissue may be from one or more of Agave spp., Alga spp., alfalfa (Medicago sativa), Arabidopsis thaliana, banana (Musa spp.), bamboo (Phyllostachys spp., Bambusa spp., Pseudosasa spp.), barley (Hordeum spp.) Brassica spp., broccoli (Brassica oleracea var. italica), cabbage (Brassica oleracea var. capitata), Cannabis spp., carrot (Daucus carota), Carrizo Cane (Arundo donax), cassava (Manihot esculenta), castor (Ricinus communis), cauliflower (Brassica oleracea var. botrytis), celery (Apium graveolens), chickpea (Cicer arietinum), Chinese cabbage (Brassica rapa), coconut (Cocos nucifera), coffee (Coffea arabica; Coffea canephor;), corn (Zea mays), clover (Trifolium spp.), cotton (Gossypium sp.), cucumber (Cucumis sativus), Douglas fir (Pseudotsuga menziesii), eggplant (Solanum melongena), eucalyptus (Eucalyptus globulus), field mustard (Brassica rapa), false flax (Camelina spp.), flax (Linum usitatissimum), garlic (Allium sativum), grape (Vitis spp.), hops (Humulus spp,), ice plant (Mesembryanthemum crystallinum), Jatropha (Jatropha curcas), leek (Allium porrum), lettuce (Lactuca sativa), millets (Panicum miliaceum), oat (Avena spp.), olive (Olea spp.), onion (Allium spp.), Opuntia spp., palm (Arecaceae spp.), pasture grass (Holcus spp.), pea (Pisum sativum), peanut (Arachis hypogaea), pepper (Capsicum spp.), potato (Solanum tuberosum), radish (Raphanus sativus), rapeseed/canola (Brassica napus), rice (Oryza spp., Zizania spp., Porteresia spp.), rye (Secale cereale), sorghum (Sorghum bicolor), soybean (Glycine max), spinach (Spinacia oleracea), squash (Cucurbita spp.), strawberry (Fragaria spp.), sugar beet (Beta Vulgaris), sugarcane (Saccharum spp.), sunflower (Helianthus spp.), sweet gum (Liquidambar styraciflua), sweet potato (Ipomoea batatas), switchgrass (Panicum spp.), tea (Camellia spp.), tobacco (Nicotiana spp.), tomato (Solanum lycopersicum), triticale (xTriticosecale), grass (Poaceae spp.), watercress (Nasturtium officinale), watermelon (Citrullus lanatus), and wheat (Triticum spp.).

In some embodiments, the plant, plant cell, plant seed, or other plant issue is from one or more of tobacco, Arabidopsis thaliana, rice, ice plant, field mustard, wheat, and barely.

the plant, the plant cell, the plant seed, or the plant tissue is one or more selected from the group consisting of tobacco, Arabidopsis thaliana, rice, ice plant, Brassica spp., field mustard, wheat, and barely.

Following transformation of target tissues, expression of, for example, the above-described selectable marker genes allow for preferential selection of transformed cells, tissues, and/or plants, using regeneration and selection methods well known in the art.

### Characterization of Arginine-Functionalized nHA Nanoparticles

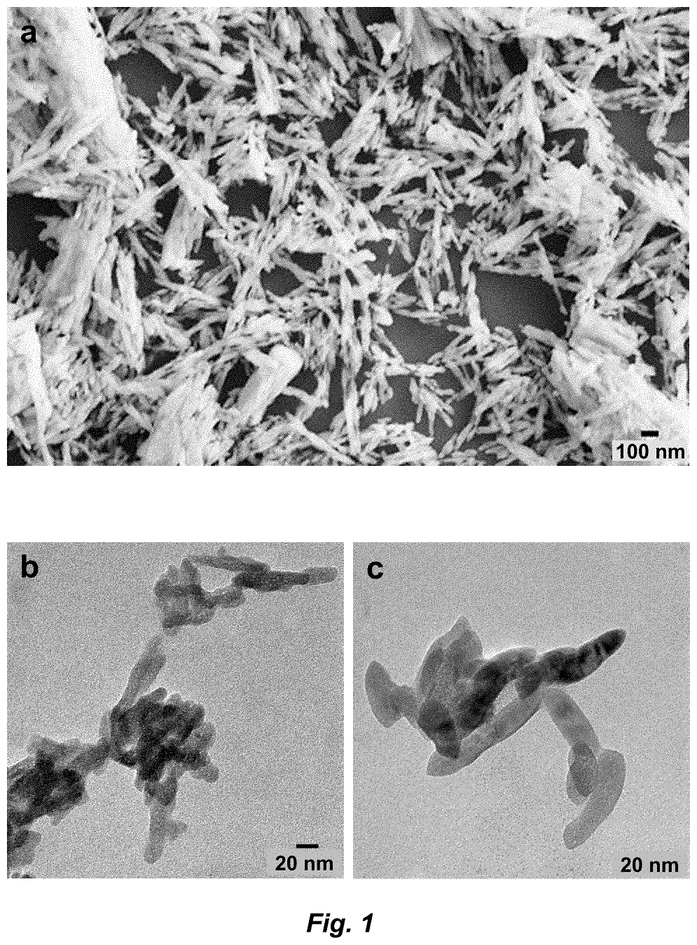

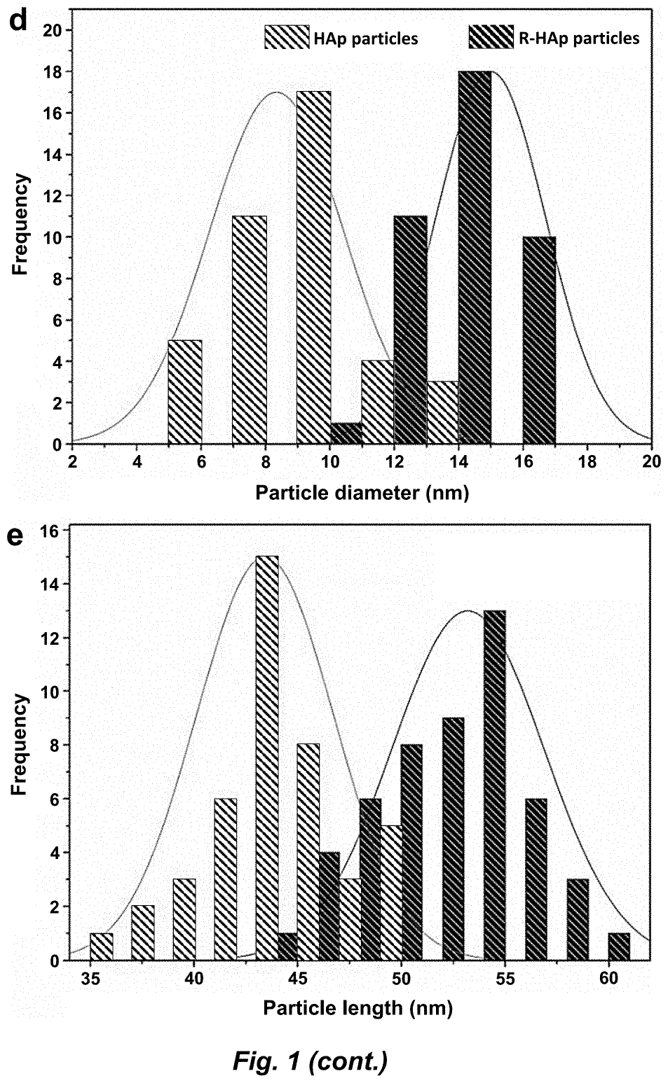

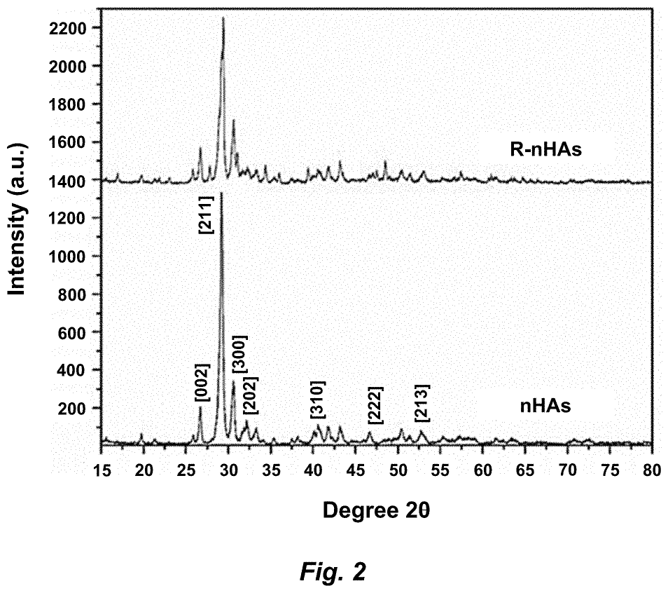

Nanohydroxyapatite rods were synthesized to carry genetic cargo for passive uptake and in planta transformation. Because the size exclusion limit of plant cell walls is ˜5-20 nm, we synthesized ˜10-nm diameter nHAs following the method of Huang et al. ⁴³ The sizes and morphologies of these nHAs were confirmed by SEM and TEM. SEM and TEM imaging confirmed that we achieved thin-rod morphologies with an average diameter of 8.5 nm and an average length of 42 nm (FIG. 1a-c). These nHAs were then functionalized with arginine (R) to enhance the electrostatic interactions between the nanoparticles and plasmid DNA (pDNA) for improved pDNA-nHA conjugate formation. The nanohydroxyapatite particles functionalized with arginine (R-nHAs) exhibited slight changes in average diameters from 8.5 nm to 15 nm and average lengths of from 42 nm to 55 nm, respectively (FIG. 1c). The surface zeta potential measurements changed slightly from −19.9 mV±2.3 mV for nHAs to −12.8 mV±1.6 mV for nHAs functionalized with arginine. This increase was attributed to the introduction of the positively charged arginine amino and guanidinium groups to the particle surfaces. Furthermore, the powder X-ray diffraction (PXRD) patterns of the synthesized nHAs showed diffraction characteristic of nHA nanorods (FIG. 2a). PXRD spectra illustrated that both the nHAs and R-nHAs have diffraction peaks at 2Θ values of 26.2, 29.9, 31.2, 34.1, 40, 46.4, and 53, which are attributable to the (002), (211), (300), (202), (310), (222), and (213) diffraction planes of the hydroxyapatite phase, respectively. These diffraction peaks confirmed the formation of hydroxyapatite nanocrystals. The patterns of the PXRD peaks for the R-nHAs remained unchanged after functionalization. The surface calcium/phosphate ratio of 1.71 for the synthesized nHAs determined by Energy Dispersive Spectroscopy (EDS) was found to be 1.71, which is close to the theoretical value of 1.66 for hydroxyapatite (Table 1).

TABLE 1
Energy Dispersive Spectroscopy properties of
hydroxyapatite nanoparticles.

| Element | Atomic % | Ca/P |
|---|---|---|
| O | 75.10 | 1.71 |
| P | 9.19 | |
| Ca | 15.71 | |

### Conjugation of Plasmid DNA to R-nHA

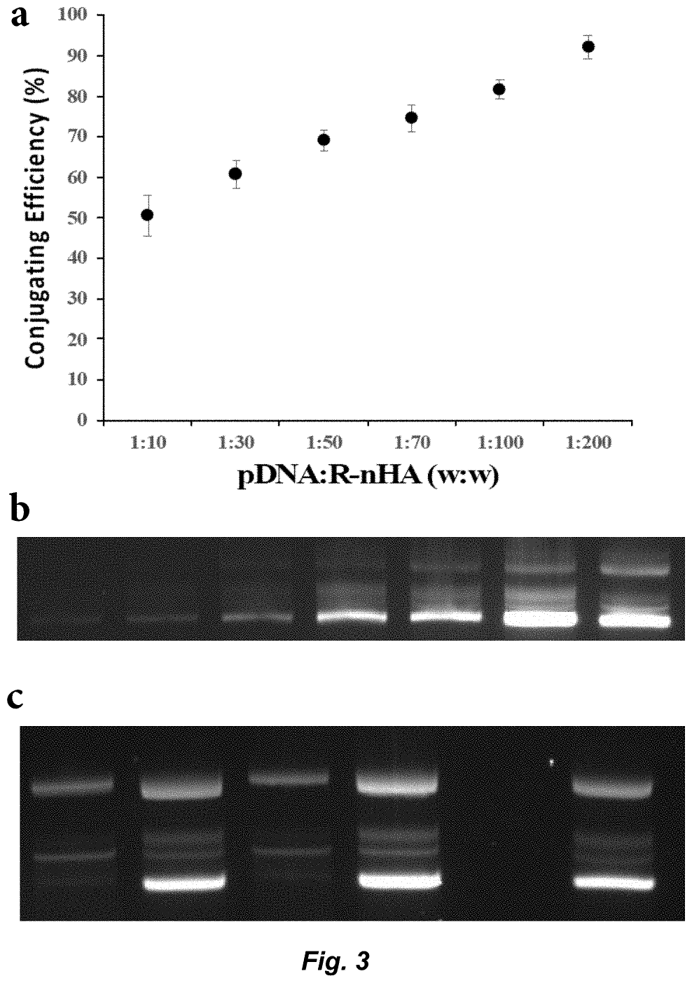

To determine the ability of pDNA to conjugate with R-nHA, we investigated the conjugating efficiency (CE) of test pDNAs to R-nHA by generating pDNA-nanoparticle conjugates with various mass ratios (w:w) ranging from 1:10 to 1:200. The conjugates were centrifuged to collect the supernatant containing the unbound pDNA. The amount of the unbound pDNA in the supernatant was then determined by measuring the optical density of the supernatant at 260 nm. CE was highest for the conjugates with 1:100 and 1:200 mass ratios (FIG. 3a). Conjugate formation was further confirmed by subjecting the recovered pellets from different ratios to gel agarose electrophoresis (FIG. 3b). To assess the stability of the pDNA-nanoparticle conjugates, we treated the 1:200 ratio pDNA conjugates to R-nHA with DNase 1 endonuclease. The test pDNAs conjugated well to R-nHA and the relaxed forms of the pDNA in particular were also partly protected from the DNAse 1 endonuclease, suggesting that nHAs might reduce or inhibit endonuclease activity (FIG. 3c).

R-nHA-Mediated Delivery of DNA Plasmid into Mature Leaves

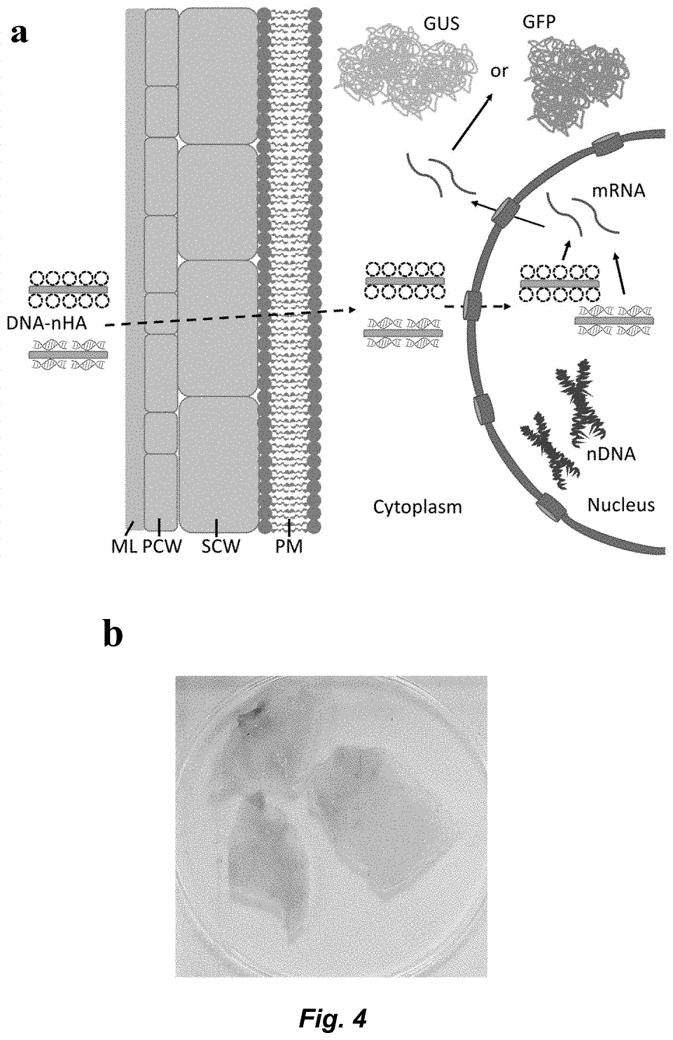

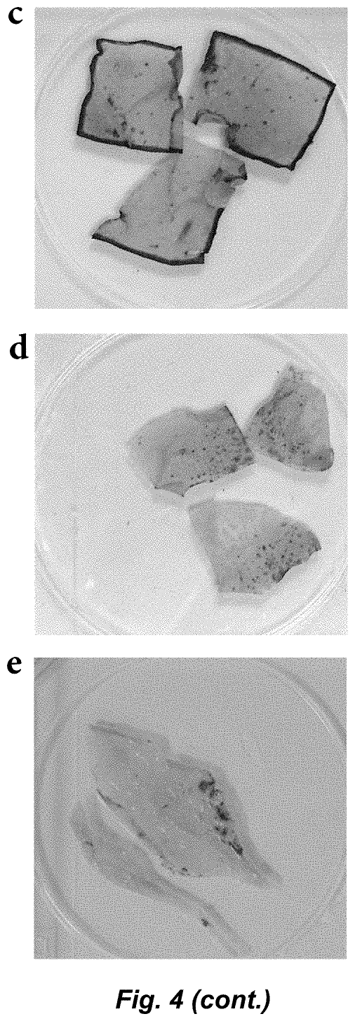

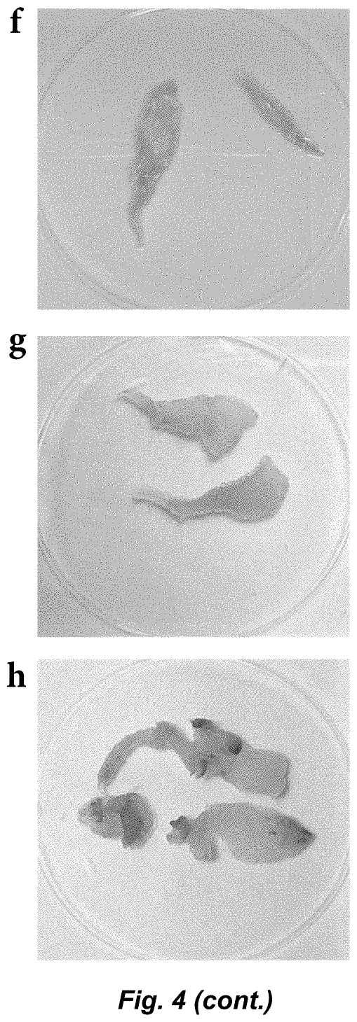

To demonstrate in vivo delivery of pDNA and transient expression in leaves using R-nHA carriers, mature leaves of Arabidopsis, tobacco, and common ice plant were infiltrated with a solution of pDNA-R-nHA conjugates. We tested the efficacy of transformation with pDNA-R-nHA conjugates in these plant species in order to demonstrate the utility of our system for delivering plasmids into a wide variety of model, and other plant species. Solutions of pDNA-R-nHA conjugates carrying a GUS-encoding reporter gene were injected into intact mature leaves using a syringe or infiltrated into detached leaves in a dish under vacuum. We hypothesized that the pDNA-R-nHA conjugates introduced into leaves would traverse the cell wall and travel into the nucleus where the GUS reporter gene carried on the pDNA would be expressed (FIG. 4a). After 2-3 d, leaves were assayed by histochemical staining for GUS followed by clearing of chlorophyll using ethanol. Transient GUS expression was observed in the leaves as characteristic blue spots or patches within leaf tissues (FIG. 4d-h).

### R-nHA-Mediated Transformation of Seeds

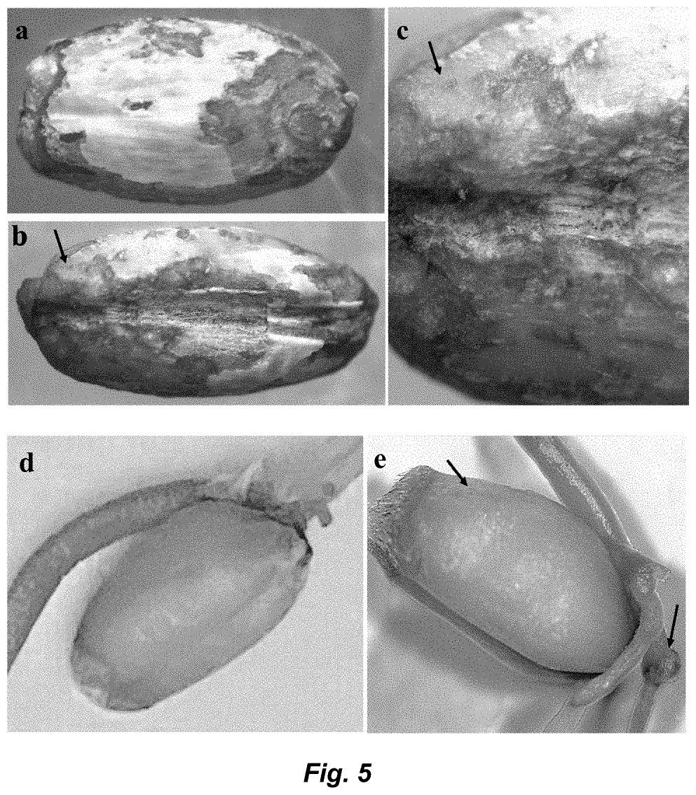

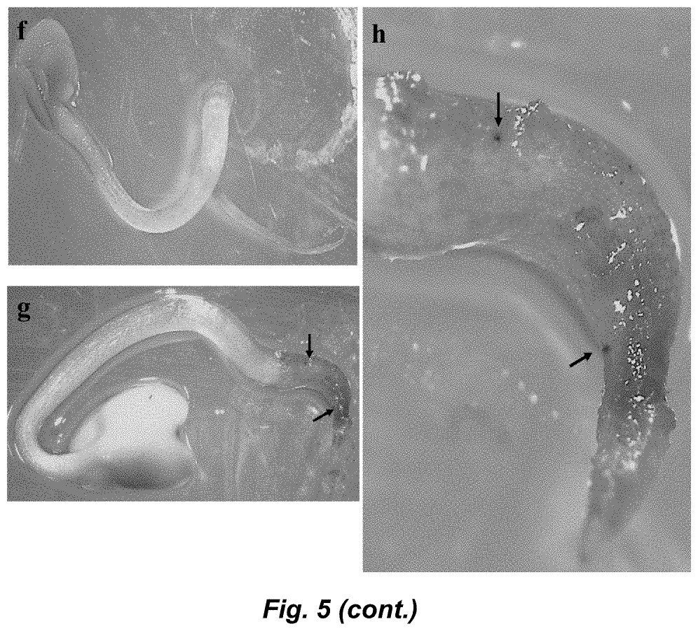

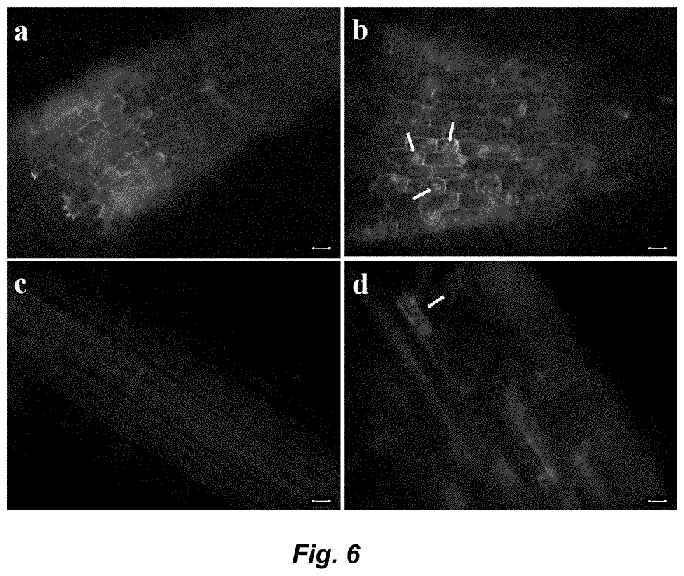

We incubated seeds of field mustard, wheat, and barley in solutions of pDNA-R-nHA conjugates and then compared transient reporter gene expression in seeds of eudicot field mustard to those of monocot wheat and barley to ensure that the pDNA-R-nHA conjugates could transform germinating seeds and to demonstrate the applicability of our system for genetic transformation of seeds of crop species from both of these paraphyletic groups. At 4 d after a 1-h incubation in a solution of pDNA-R-nHA conjugates, GUS protein expression was confirmed by examining seeds under a dissecting microscope after histochemical staining for GUS activity. GUS protein was expressed in all seeds subjected to the transformation protocol, and most interestingly in the developing embryonic tissues of wheat, and field mustard (FIGS. 5a and h). In addition, GFP protein expression was observed in the cytoplasm as green patches within root cells as predicted (FIG. 4a) in both Arabidopsis and field mustard seedlings (FIG. 6a-d).

GUS expression was not observed in the developing embryonic tissue of barley, as it takes more than six days to germinate, and thus transient GUS expression would not be observable when germination finally occurs. The expression of GUS activity in developing embryonic tissues of wheat, barley and field mustard suggest that our system for delivery of genetic cargo has very high potential for stable genetic transformation of germline tissues in seeds or even in reproductive organs. Altogether, our R-nHA-based plasmid DNA delivery method enables passive delivery of DNA into both leaf cells and germinating seeds of several plant species with high efficiency and no observable adverse effects on germination.

We have demonstrated in planta genetic transformation of tissues in mature plant leaves and germinating seeds using a nano-biomimetic carrier for genetic cargo. Our system uses synthesized R-nHAs as nanocarriers to allow pDNA to traverse plant cell walls and be delivered into leaf cells. By using a pDNA carrying a GUS or GFP reporter gene, we have shown successful delivery and transient heterologous gene expression in germinating seeds of Arabidopsis, wheat, barley, field mustard, as well as in detached and intact mature leaves of Arabidopsis, tobacco, and common ice plant. This passive transformation system has all the advantages of other nanoparticle-mediated approaches including that it is simple, rapid, cost-effective, nondestructive, species-independent, scalable, and optimizable, and also eliminates unwanted integration of vector sequences into the target genome. These nanocarriers also possess the further advantages of noncytotoxicity, ready biodegradability, biocompatibility, and less potential for horizontal transfer of genetic cargo.

Nanoparticle-mediated gene delivery has immense promise for advancing plant genetic engineering because nanoparticles are cell wall-permeable; however, their use has raised some concerns regarding the health of organisms and ecosystems. Notably, some engineered nanomaterials, especially carbon-based forms, are difficult to break down or can be cytotoxic to both animals and plants. Their harmful effects can include generation of reactive oxygen species, DNA damage, lysosomal damage, mitochondrial dysfunction, and eventual cell death via apoptosis or necrosis. Some engineered nanomaterials can also activate various immune cells or induce immunosuppression. Although the physicochemical properties of highly persistent engineered nanomaterials can be functionalized so that they are then less cytotoxic or noncytotoxic, their extreme stability and resistance to degradation might result in their bioaccumulation in organisms and the environment. Undeniably, one of the critical issues regarding engineered nanomaterials as gene carriers for plants is the potential for horizontal transfer of recombinant genes conjugated to the engineered nanomaterials to nontarget organisms, especially if the carrier does not readily break down. Specifically, recombinant genes conjugated to nanomaterials can be directly or indirectly transferred to nontarget plant species including weedy species. In contrast, because nHA-based nanomaterials are readily broken down within plants and conjugated recombinant genes can then be digested by endogenous nucleases, horizontal transfer of recombinant genes to nontarget organisms will be much less likely. In addition, because nHAs are composed of the elements Ca and P, their broken-down components could provide an excellent form of nutrients for plants. For instance, application of nHAs or HAs as a fertilizer enhanced the germination and radicle growth of chickpea (Cicer arietinum) seeds, increased the growth rate and yield of soybean plants, and increased the growth of lettuce (Lactuca sativa L.) plants without adverse impacts on the growth of soil bacteria. Interestingly, nHAs can also reduce the mobility of lead in the rhizosphere and toxicity of lead in roots of rice. Altogether, the above characteristics of nHAs make them ideal components of a nano-biomimetic plant transformation system.

In the present study, we have developed a nHA-based nano-biomimetic gene carrier for plant transformation wherein the carrier does not remain in plant cells or disrupt cell function, and also less likelihood in horizontal transfer of heterologous genes to nontarget organisms. When combined with nuclease-based genome editing tools, the nHA-based approach does not result in the integration of gene editing components into the host genome as happens with other nanoparticle-mediated delivery approaches. Indeed, our initial results suggest that this nano-biomimetic transformation system could allow stable transformation of crop species lines without multiple rounds of selection and breeding to remove the plasmid nanocarrier as required with carbon-based nanocarriers. Hence, our nHA-based genetic transformation system could enable more rapid release of transformed plants from lab to field for analysis and various applications. Our results indicate that the in planta nano-biomimetic plant transformation system we have developed is a sound nanoparticle-mediated approach that should be useful for diverse biotechnological and agricultural applications in plant biology and crop science.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1. Materials and Methods

### Synthesis of Nanohydroxyapatites

Rod-shaped nanohydroxyapatites (nHAs) 10 nm in diameter were synthesized following Huang et al., J. Mater. Sci., 2007, 42, 8599-860. To minimize particle aggregation, the nHAs were prepared in the presence of the nontoxic, nonionic biocompatible surfactant PEG 300 as a chemical dispersant. (Liu et al., Sci. Rep., 2014, 4, 5686). An aliquot of PEG-300 (1336 ml) was added by micropipette to a 100-ml volumetric flask and the volume was brought to 100 ml with deionized water to prepare a 1.5% (w/w) aqueous solution of PEG. The prepared PEG solution was mixed with 0.5549 g of CaCl₂ to prepare 100 ml of CaCl₂ solution (0.05M, 100 ml). The resulting solution was equilibrated overnight (about 12 h) at room temperature (RT) and then added dropwise (1.6 ml/min) into 100 mL of 0.03 M aqueous Na₂HPO₄ solution with mechanical stirring at a constant speed of 1000 rpm. The resulting solution was placed in a closed glass vial and incubated for 48 h at RT. After 48 h, the solution was centrifuged at 11,000 rpm for 10 min to separate the white precipitate. The separated white precipitate was washed three times with deionized water (Thermo Scientific™, Barnstead™ Nanopure), and then three times with absolute ethanol, and finally dried at 60° C. overnight to obtain the as-prepared powder product, which was then calcined at 500° C. for 2 h to produce the nHAs.

### Arginine-Functionalized Nanohydroxyapatites

Nanohydroxyapatites (100 mg) were functionalized following the procedure of Deshmukh et al., Mater. Sci. Eng. C. Mater. Biol. Appl., 2019, 96, 58-65 with a slight modification. Briefly, nHAs were functionalized by mixing them with a solution of positively charged arginine (100 ml, 0.1 wt %). The mixture was stirred at 600 rpm for 1 h. The functionalized particles were separated and washed three times with deionized water followed by centrifugation at 8000 rpm for 10 min. The washed particles were dried at ambient temperature overnight (about 12 h) to obtain about 310 mg of arginine-functionalized nHAs (R-nHAs).

### Scanning Electron Microscopy

A 5-mg sample of the synthesized nHAs was dispersed in ethanol (10 ml) using an ultrasonication probe. One droplet of the dispersion was deposited onto the reflective face of a silicon wafer and dried at 600° C. for 30 min for SEM imaging. Particle sizes and shapes were imaged using a Zeiss Sigma Field Emission Scanning Electron Microscope (SEM) with an accelerating voltage of 4 kV (Carl Zeiss Microscopy, LLC, White Plains, N.Y. USA).

### Transmission Electron Microscopy

Synthesized nHAs (1 mg) or R-nHAs (1 mg) were dispersed in 20 ml of deionized water and one droplet of each suspension was deposited onto copper-coated TEM grids for imaging and air-dried for 2 d in a vacuum desiccator. Nanoparticle morphologies were imaged using a FEI Tecnai G2 TWIN TEM with an accelerating voltage of 120 kV (Field Electron and Ion Company, Hillsboro, Oreg., USA).

### Powder X-Ray Diffraction

High-angle powder X-ray diffraction (PXRD) patterns for nHAs and R-nHAs were recorded on a Rigaku Ultima IV XRD Diffractometer (Rigaku Analytical Devices, Inc., Wilmington, Mass., USA) using Cu Kα radiation (λ=1.54 A°, 40 kV, 44 mA). The diffraction spectra were collected over a range of 15° to 80° with a 0.02° sampling width.

### Zeta Potential Measurement

The surface zeta potential measurements of nHAs and R-nHAs (before and after arginine functionalization) were performed on a Malvern Zetasizer ZS Nano S instrument (Malvern Panalytical Ltd., Malvern UK).

Energy Dispersive Spectroscopy analysis

For the energy dispersive spectroscopy (EDS) analysis, a small amount of nHA powder was placed on self-adhesive carbon tape and sputter-coated with 28 nm of carbon using a Denton Desk V TSC sputter coating accessory (Denton, Moorestown, N.J. USA). An Oxford Instruments X-MaxN 50 EDS probe (Oxford Instruments NanoAnalysis, Concord, Mass., USA) was used to conduct elemental analysis of prepared nHAs with the probe attached to a ZEISS Sigma Field.

### Plant Growth

Arabidopsis thaliana, ice plant, and tobacco plants were grown in Miracle-Gro® Moisture Control® potting mix under a 16-h day:8-h night photoperiod at ˜22° C. in an environmentally controlled room. Barley seeds and wheat seeds were obtained from Amazon.com, and field mustard seeds were obtained from toddsseeds.com.

Cloning and Plasmid Isolation of pGWB402::GUS

The gene encoding β-glucuronidase (GUS) with flanking attL and attR sites was synthesized by Gene Universal (Newark, Del., USA). The pGWB402::GUS construct was generated using the LR cloning reaction with pGWB402 expression vector and the synthesized GUS-encoding gene. The resulting plasmid pGWB402::GUS was transformed into 10-beta competent Escherichia coli cells (New England Biolabs, Ipswich, Mass., USA). Plasmid DNA was extracted from the E. coli cells using the Qiagen Plasmid Mini Kit (Qiagen, Hilden, Germany). The 11,833 bp pGWB402::GUS vector is referred to as pDNA herein.

Preparation of pDNA-nHA and pDNA-R-nHA Conjugate

R-nHA and the pDNA carrying the reporter gene encoding β-glucuronidase (GUS) was used to prepare pDNA-R-nHA conjugates following the procedure of del Valle et al., J. Mater. Chem. B, 2014, 2, 6953-6966 with modifications. Briefly, an aqueous suspension of 1 mg ml⁻¹ of R-nHA was evenly dispersed by sonicating in an ice bath for 10 min. One μg of pDNA and 200 μg of R-nHA was used to make a 1:200 pDNA:R-nHA w:w ratio conjugate mixture. The 1:200 pDNA:R-nHA ratio conjugate mixture was then thoroughly mixed by vortexing for 30 sec. After vortexing, the mixture was incubated at 37° C. with shaking at 200 rpm for 90 min. Unbound pDNA was removed by centrifugation at 10,000 rpm for 10 min. The precipitate was resuspended in 10 ml of 0.5% low-viscosity carboxymethylcellulose (CMC) and stirred for 2 h at RT to keep the pDNA-R-nHA conjugates in suspension. The formation of pDNA-R-nHA conjugates was visualized on 1% agarose gel.

pDNA Conjugating Efficiency

The efficiency of pDNA conjugating to R-nHA was assessed following the procedure of Deshmukh et al., Mater. Sci. Eng. C. Mater. Biol. Appl., 2019, 96, 58-65. Briefly, aqueous 1 mg ml⁻¹ suspensions of R-nHA were prepared by sonicating for 10 min on ice. An aliquot 500 ng of pDNA was mixed with R-nHA suspensions at different w:w ratios (1:10, 1:30, 1:50, 1:70, 1:100, 1:200). The mixtures were shaken at 37° C. for 10 min at 200 rpm and centrifuged at 15,000 rpm for 4 min. The supernatant containing the unbound pDNA was used to determine the conjugating efficiency of R-nHA to pDNA. The optical density of the supernatant was determined at 260 nm relative to the supernatants from R-nHA without pDNA as blanks. The DNA conjugating efficiencies (CEs) were determined using the equation:

% CE={[(pDNA)i−(pDNA)f]/[(pDNA)i]}×100,

where % CE=percent conjugating efficiency, [pDNA]i=the optical density of the initial amount of pDNA added to the reaction mixture, and [pDNA]f=the optical density of the unbound pDNA remaining in the sample. The assay was independently repeated three times with three technical replicates per assay. The pellets were dissolved in 50 μl of DNase/RNase free water and 10 μl of the pDNA-R-nHA conjugates were separated on 1% agarose gel to confirm conjugation of pDNA to R-nHA.

### Nuclease Digestion

The 1:200 ratio (w:w) pDNA-R-nHA conjugates were prepared as described above, and 100-ng aliquots of the precipitates were resuspended in 20 ul of Dnase/RNase free water, respectively. DNasel (250 U/μl) (Zymo Research, Tustin, Calif. USA) was added (1 μl) to the resuspended pDNA-R-nHA conjugates and digestion allowed to proceed for 1 h at RT. After incubation, samples were mixed with 2 μl of 6× loading dye (New England Biolabs Ltd., Ipswich, Mass. USA) and loaded onto 1% w/v agarose for electrophoresis. Samples of digested and undigested pDNA or undigested pDNA-R-nHA were used as controls.

### Agrobacterium-Mediated Transformation

Agrobacterium-mediated transformation was used as a positive control for in vivo transient expression infiltrations using the pDNA-R-nHA conjugates. The vector pGWB402::GUS was transformed into Agrobacterium strain GV3101 using the freeze-thaw method of Wise et al. ⁴⁶ A single Agrobacterium colony was inoculated into liquid Luria-Bertani (LB) medium containing the appropriate antibiotics for binary vector selection. This feeder culture was incubated at 28° C., 250 rpm for 2 d. Then the feeder culture was centrifuged at 3000 rpm for 10 min at RT and the supernatant discarded. The pellet was resuspended in infiltration buffer (10 mM MES, 10 mM MgCl₂, pH=5.6). Centrifugation and resuspension steps were repeated three times to remove any remaining LB to stop Agrobacterium growth. The bacterial pellet was resuspended in the infiltration buffer to obtain a 1:10 dilution (OD₆₀₀ ˜0.5) and acetosyringone was added to the infiltrate to a final concentration of 200 μM. Tobacco leaves were infiltrated using the syringe infiltration method of Leuzinger et al., J. Vis. Exp., 2013, 77, 50521 and incubated for 3 d in an environmentally controlled room before performing GUS assays.

In Planta pDNA-R-nHA Leaf Infiltration

A 1:200 (w:w) pDNA-R-nHA conjugate was used for plant leaf infiltration. Briefly, the pDNA-R-nHA mixture was shaken for 90 min at 200 rpm at 37° C. Unbound pDNA was removed by centrifugation at 10,000 rpm for 10 min. The precipitate was resuspended in 10 ml of aqueous 0.5% low-viscosity CMC and stirred for 2 h at room temperature to keep the R-nHA particles suspended. (Liu et al., Sci. Rep., 2014, 4, 5686) The formation of pDNA-R-nHA conjugates was visualized on 1% agarose gel. Mature A. thaliana and tobacco leaves were infiltrated using a syringe and left for 3 d in an environmentally controlled room prior to performing GUS assays.

Healthy leaves of A. thaliana, common bean, and ice plant were detached from whole plants and transferred into Petri dishes containing pDNA-R-nHA suspended in 0.5% CMC. The solution was vacuum infiltrated into the leaves at −0.01 MPa for 1 min following the procedure of Leuzinger et al., J. Vis. Exp., 2013, 77, 50521 and the process was repeated twice before transferring the dishes containing the infiltrated leaves to an environmentally controlled room. The leaves were kept moist in water for 4 d prior to assaying for GUS expression.

Incubation of Seeds in a Solution of pDNA-R-nHA Conjugates

Seeds of common wheat, field mustard, and barley were pre-soaked in water (i.e., control), or in a solution of pDNA-R-nHA conjugates for 15 min and then vacuum infiltrated at −0.01 mPA for 1 min. Vacuum infiltration was repeated three times at 15 min intervals. The common wheat, and barley seeds were kept moist for 4 d before testing for expression of the gene encoding GUS, whereas field mustard seeds were kept moist for 6 d to allow the seeds to sprout before testing for expression of the gene encoding GUS. Germinated seeds of Arabidopsis and field mustard were also pre-soaked in water (i.e., control), or in a solution of pDNA-R-nHA conjugates, where the pDNA was a vector carrying a G3 green fluorescent protein gene (i.e., G3GFP in pGWB542). GFP fluorescence was visualized after 3 d using a Lecia DMRA2 fluorescence microscope with a Leica DFC3000 G camera (Leica Microsystems Inc., Buffalo Grove, Ill.).

### GUS Assay

The GUS assay solution was prepared following the procedure of Lim et al. by dissolving 0.5 mg/ml of X-Gluc (5-bromo-4-chloro-3-indolyl glucuronide) in GUS assay buffer (50 mM sodium phosphate buffer, pH 7.0; 0.1 mM K₄Fe(CN)₆; 0.1 mM K₃Fe(CN)₆; and 4 mM EDTA). The GUS assay solution was introduced into the seeds by vacuum infiltration at −0.07 MPa for 10 min. The infiltrated seeds were incubated for 36 h at 37° C. and chlorophyll removed in 100% ethanol overnight before images were captured using a Lecia EZ24 HD Stereo Microscope (Leica Microsystems Inc., Buffalo Grove, Ill.).

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A nanoparticle comprising a hydroxyapatite core and a polynucleotide attached to a surface of the hydroxyapatite core;
wherein the nanoparticle is rod-shaped, having an average diameter of about 2 nm to about 20 nm and an average length of about 20 nm to about 70 nm.

2. The nanoparticle of claim 1 wherein average diameter is about 5 nm to about 15 nm and the average length is about 35 nm to about 55 nm.

3. The nanoparticle of claim 2 wherein average diameter is about 7 nm to 12 nm and the average length is about 40 nm to about 50 nm.

4. The nanoparticle of claim 3 wherein average diameter is about 8.5 nm and the average length is about 42 nm.

5. The nanoparticle of claim 1 wherein the polynucleotide is bonded to the surface of the nanoparticle through ionic interaction.

6. The nanoparticle of claim 1 wherein the surface of the hydroxyapatite is functionalized with a positively charged amino acid, and the polynucleotide is ionically bonded to the positively charged amino acid.

7. The nanoparticle of claim 6 wherein the positively charged amino acid is arginine.

8. The nanoparticle of claim 7 wherein the average diameter is about 10 nm to about 18 nm and the average length is about 45 nm to about 60 nm.

9. The nanoparticle of claim 8 wherein average diameter is about 12 nm to about 17 nm and the average length is about 50 nm to about 57 nm.

10. The nanoparticle of claim 9 wherein average diameter is about 15 nm and the average length is about 55 nm.

11. A composition comprising the nanoparticle of claim 1 and pharmaceutically acceptable carrier.

12. A rod-shaped nanoparticle consisting of:
hydroxyapatite;
a positively charged amino acid disposed on a surface of the hydroxyapatite; and
a polynucleotide ionically conjugated to the positively charged amino acid, wherein the nanoparticle is about 8.5 nm to about 15 nm in diameter and about 42 nm to about 55 nm in length.

13. A composition comprising:
a rod-shaped hydroxyapatite nanoparticle and a polynucleotide attached to a surface of the hydroxyapatite nanoparticle, and
a pharmaceutically acceptable carrier comprising water;
wherein the hydroxyapatite nanoparticle is about 8.5 nm to about 15 nm in diameter and about 42 nm to about 55 nm in length.

14. The composition of claim 13 wherein the pharmaceutically acceptable carrier further comprises a thickening agent present in a concentration of about 0.1% w/v to about 1% w/v, the thickening agent selected from the group consisting of carboxymethyl cellulose, methyl cellulose, hydroxyethyl cellulose, and hydroxypropylmethylcellulose; and the pharmaceutically acceptable carrier optionally includes one or more buffers, salts, or a combination thereof.

15. The composition of claim 14 wherein the thickening agent is carboxymethyl cellulose present in a concentration of about 0.5% w/v.

16. The composition of claim 13 wherein the surface of the hydroxyapatite nanoparticle is functionalized with an arginine linker, and the polynucleotide is ionically bonded to the arginine linker.

17. A method of genetically transforming a cell comprising contacting the cell with the nanoparticle of claim 1 wherein the nanoparticle passes through a cell membrane of the cell and is transported to a nucleus, and a heterologous gene of the polynucleotide is expressed to transform the cell.

18. A method for producing a genetically modified plant comprising contacting a plant, a plant cell, a plant seed, or plant tissue with a nanoparticle functionalized with a polynucleotide, the nanoparticle comprising a rod-shaped hydroxyapatite core particle and the polynucleotide is ionically bonded to a surface of the nanoparticle, and the nanoparticle passing through a cell wall of the plant cell and is transported to a plant nucleus such that a heterologous gene of the polynucleotide is expressed.

19. The method of claim 18 wherein the plant, the plant cell, the plant seed, or the plant tissue is one or more of alfalfa, Arabidopsis, banana, barley, bean, broccoli, cabbage, carrot, cassava, castor, cauliflower, celery, chickpea, Chinese cabbage, coconut, coffee, corn, clover, cotton, cucumber, Douglas fir, eggplant, eucalyptus, flax, garlic, grape, hops, leek, lettuce, millets, oat, olive, onion, palm, pasture grass, pea, peanut, pepper, potato, radish, rapeseed, rice, rye, sorghum, soybean, spinach, squash, strawberry, sugar beet, sugarcane, sunflower, sweet corn, sweet gum, sweet potato, switchgrass, tea, tobacco, tomato, triticale, grass, watermelon, and wheat.

20. The method of claim 17 wherein the plant, the plant cell, the plant seed, or the plant tissue is one or more selected from the group consisting of tobacco, Arabidopsis thaliana, rice, ice plant, field mustard, wheat, and barely.
